(19) United States
(12) Patent Application Publication
(10) Pub. No.: US 2015/0238119 A1
(43) Pub. Date: Aug. 27, 2015

(54) BREATH SAMPLING TUBES WITH ARRAY OF RESERVOIRS

(71) Applicant: ORIDION MEDICAL 1987 LTD., Jerusalem (IL)

(72) Inventors: Joshua Lewis Colman, Jerusalem (IL); Alon Sasson, Rehovot (IL); Paul S. Addison, Midlothian (GB); Avner Bar-Lev, Givatayim (IL); Seamus Maguire, Athlone (IE); Michal Ronen, Givat Brenner (IL)

(73) Assignee: ORIDION MEDICAL 1987 LTD., Jerusalem (IL)

(21) Appl. No.: 14/192,069

(22) Filed: Feb. 27, 2014

(51) Int. Cl. A61B 5/097; A61B 5/08

(52) U.S. Cl. CPC A61B 5/097; A61B 5/082

(57) ABSTRACT

Breath sampling tube having an array of reservoirs, the array of reservoirs including a first reservoir proximate to a device end of the tube and at least one intermediate reservoir; the first reservoir having an inner wall defining a channel, a hydrophobic element and a hydrophilic element; and the at least one intermediate reservoir having an inner wall defining a channel and a hydrophilic element; wherein the array of reservoirs is configured to allow unabsorbed water vapor to proceed through the at least one intermediate reservoir while essentially blocking unabsorbed water vapor from passing through the first reservoir.

### TECHNICAL FIELD

The present disclosure generally relates to the field of breath sampling tubes.

### BACKGROUND

Accurate monitoring concentrations of a gas, such as for example carbon dioxide (CO₂) in exhaled breath is vital in assessing the physiologic status of a patient. Breath sampling is generally performed through breath sampling tubes configured to be connected to a patient airway and to a medical device.

Liquids are common in patient sampling systems, and have several origins, for example condensed out liquids from the highly humidified air provided to and exhaled from the patient. These liquids typically accumulate both in the patient airway and in the sampling line tubing; secretions from the patient, typically found in the patient airway; and medications or saline solution provided to the patient during lavage, suction and nebulization procedures.

### SUMMARY

The present disclosure relates to breath sampling tubes including an array of reservoirs. The breath sampling tubes disclosed herein are configured to evaporate liquids.

One of the major obstacles when designing a filter system is the necessity to prevent any liquids from blocking the breath sampling path and even more importantly from reaching the measurement sensor, while providing continuous, smooth, undisturbed sampling of the patient's breath.

A well-known problem with gas sampling lines is that they may eventually saturate allowing the line to become clogged. Lines are designed so that water vapor is captured and evaporated through the tube surface. At high humidity, however, the evaporation flow rate may be less than the capture rate and so eventually the reservoir, or other suitable liquid collection element, saturates and the line may become clogged. The time it takes for this to happen is known as the lifetime of the line.

The breath sampling tubes disclosed herein include an array of reservoirs. The array of reservoirs includes a first reservoir proximate to a device end of the tube and at least one intermediate reservoir. The first reservoir includes a hydrophobic element and a hydrophilic element whereas the at least one intermediate reservoir includes a hydrophilic element. The array of reservoirs is configured to allow unabsorbed water vapor to proceed through the at least one intermediate reservoir along the tube while essentially blocking the unabsorbed water vapor from passing through the first reservoir. Accordingly, the evaporation of liquids from the tube surface is enhanced and hence the lifetime of the line extended.

The number of reservoirs in the array may be modular. This may advantageously facilitate breath sampling lines having different lifetimes accommodating the intended use of the line. For example, breath sampling tubes for use with patients requiring long term monitoring of their respiratory gases, may include a relatively large number of intermediate reservoirs, whereas sampling lines for temporary measurements may include only one or a few intermediate reservoirs.

The array of reservoirs may be arranged such as to influence the rigidity of the tube and the ease of manufacturing. For example, the tubes may bend in regions distributed between the intermediated reservoirs.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

According to some embodiments, there is provided a breath sampling tube comprising an array of reservoirs, the array of reservoirs comprising a first reservoir proximate to a device end of the tube and at least one intermediate reservoir.

According to some embodiments, the first reservoir includes an inner wall defining a channel, a hydrophobic element and a hydrophilic element.

According to some embodiments, the at least one intermediate reservoir includes an inner wall defining a channel and a hydrophilic element. According to some embodiments, the array of reservoirs is configured to allow unabsorbed water vapor to proceed through the at least one intermediate reservoir while essentially blocking unabsorbed water vapor from passing through the first reservoir. According to some embodiments, the at least one intermediate reservoir is devoid of a hydrophobic element. According to some embodiments, the hydrophobic element of said first reservoir is disposed across said channel of said first reservoir at a proximal end thereof.

According to some embodiments, the hydrophilic elements of the first and intermediate reservoirs are coextensive with the inner wall of the first and intermediate reservoirs. According to some embodiments, the hydrophilic elements include a hydrophilic material transiting the inner wall. According to some embodiments, the hydrophilic material is of a thickness that maximizes water vapor while minimizing disruption of breath flow in said tube. According to some embodiments, the hydrophilic elements of the first and intermediate reservoirs align an inner side of the inner walls of the first and intermediate reservoirs.

According to some embodiments, the inner walls include a plurality of port holes configured to allow breath, flowing in the channel of the first reservoir and of the intermediate reservoir, to access the hydrophilic elements.

According to some embodiments, the array of reservoirs is attached to, embedded in or molded on said tube.

According to some embodiments, the array of reservoirs includes at least two intermediate reservoirs disposed serially along the tube. According to some embodiments, the array of reservoirs includes at least two intermediate reservoirs disposed parallel along the tube.

According to some embodiments, the array of reservoirs includes a plurality of intermediate reservoirs. According to some embodiments, the plurality of intermediate reservoirs is disposed serially and/or parallel along the tube.

According to some embodiments, the at least one intermediate reservoir is configured to reduce the amount of water vapor reaching the first reservoir. According to some embodiments, the array of reservoirs enhances the evaporation of liquids from the tube, thereby extending the life time of the tube.

According to some embodiments, the breath sampling tube further includes an inner conduit. According to some embodiments, the inner conduit is configured to permit gas flow along a central portion of the conduit and to store liquids along a surface of the conduit. According to some embodiments, the surface of said inner conduit comprises a hydrophilic material.

According to some embodiments, there is provided a breath sampling system including: a breath sampling tube having an array of reservoirs and at least one connector. According to some embodiments, the array of reservoirs includes a first reservoir proximate to a device end of the tube and at least one intermediate reservoir. According to some embodiments, the first reservoir includes an inner wall defining a channel, a hydrophobic element and a hydrophilic element. According to some embodiments, the at least one intermediate reservoir includes an inner wall defining a channel and a hydrophilic element. According to some embodiments, the array of reservoirs is configured to allow unabsorbed water vapor to proceed through the at least one intermediate reservoir while essentially blocking unabsorbed water vapor from passing through the first reservoir.

According to some embodiments, there is provided a method including forming a breath sampling tube having an array of reservoirs, the array of reservoirs including a first reservoir proximate to a device end of the tube and at least one intermediate reservoir. According to some embodiments, the first reservoir includes an inner wall defining a channel, a hydrophobic element and a hydrophilic element. According to some embodiments, the at least one intermediate reservoir includes an inner wall defining a channel and a hydrophilic element. According to some embodiments, the array of reservoirs is configured to allow unabsorbed water vapor to proceed through the at least one intermediate reservoir while essentially blocking unabsorbed water vapor from passing through the first reservoir.

According to some embodiments, at least part of the tube is formed of a material configured to evaporate water.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates the lifetime of tube lines against the vapor pressure of water (P_{H2O}) in prior art tube lines (A), and for the tube lines disclosed herein (B), according to some embodiments;

FIG. 2 schematically illustrates a first reservoir, according to some embodiments;

FIG. 3A schematically illustrates an intermediate reservoir, according to some embodiments;

FIG. 3B schematically illustrates an intermediate reservoir, according to some embodiments;

FIG. 4 schematically illustrates a breath sampling line including an array of reservoirs, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

There is provided, according to some embodiments, a breath sampling tube comprising an array of reservoirs. According to some embodiments, the array of reservoirs includes a first reservoir proximate to a device end of the tube and at least one intermediate reservoir.

As used herein, the terms “breath sampling tube”, “sampling line” and “breath sampling line” may refer to any type of tubing(s) or any part of tubing system adapted to allow the flow of sampled breath, for example, to an analyzer, such as a capnograph. The sampling line may include tubes of various diameters, adaptors, connectors, valves, drying elements (such as filters, traps, drying tubes, such as Nafion® and the like).

According to some embodiments, the term “reservoir” may refer to a trap or other suitable capture elements configured to accumulate and/or wick up water vapor or other liquids found in breath samples. According to some embodiments, the reservoir may be configured to condense out liquids through the walls thereof.

According to some embodiments, the term “hydrophobic element” may refer to filters, membranes, layers or other suitable component having the ability to repel water.

According to some embodiments, the term “hydrophilic element” may refer to filters, membranes, layers or other suitable component having affinity to water and accordingly be configured to allow therethrough or optionally to absorb water (or another hydrophilic liquid).

As used herein, the terms “distal” and “distal end” may refer to the part of the tube closest to the subject. The length of the distal end may for example be 0.5, 1, 2, 3, 4, 5, 10 cm or more. Each possibility is a separate embodiment.

As used herein, the terms “proximal” and “proximal end” may refer to the part of the tube closest to the medical device. The length of the proximal end may for example be 0.5, 1, 2, 3, 4, 5, 10 cm or more. Each possibility is a separate embodiment.

As used herein, the terms “proximity” and “proximate” to may refer to 30, 20, 15, 10, 5, 1, 0.5 cm or less. Each possibility is a separate embodiment.

As used herein, the term “certain distance” may refer to a distance larger than 10 cm, for example larger than 20 cm, 30 cm, 40 cm or 50 cm, 70 cm. Each possibility is a separate embodiment.

According to some embodiments, the first reservoir may include an inner wall defining a channel, a hydrophobic element and a hydrophilic element. According to some embodiments the at least one intermediate reservoir may include an inner wall defining a channel and a hydrophilic element. According to some embodiments, the intermediate reservoir is devoid of a hydrophobic element. According to some embodiments, the array of reservoirs is configured to allow unabsorbed water vapor to proceed through the at least one intermediate reservoir while essentially blocking unabsorbed water vapor from passing through the first reservoir. According to some embodiments, the at least one intermediate reservoir is configured to reduce the amount of water vapor reaching the first reservoir.

According to some embodiments, the first reservoir is configured to be connected adjacent to a medical device. According to some embodiments, the proximal end of the first reservoir may be connected directly to the medical device, for example via a connector molded on or otherwise attached to the reservoir at the proximate end thereof. Alternatively, the first reservoir may be connected adjacent, but indirectly, to the medical device, for example via a continuation of the breath sampling tube at the proximal end of the first reservoir.

It is understood by one of ordinary skill in the art that such configuration prevents water from reaching the medical device on the one hand, while on the other hand avoids clogging of the breath sampling tube due to accumulation of liquids in the tube.

According to some embodiments, the channels of the first and intermediate reservoirs may be coextensive with the breath sampling tube, thereby allowing undisturbed flow of breath through the channel and the tube.

According to some embodiments, the hydrophilic elements of the first and intermediate reservoirs may be coextensive with the inner wall of the first and intermediate reservoirs. This may allow liquids, present in breath flowing in the channel of the reservoirs, to be absorbed and/or condensed out by the hydrophilic elements. According to some embodiments, the hydrophilic elements may include a hydrophilic material transiting the inner wall. The hydrophilic material may be of a thickness that maximizes water vapor while minimizing disruption of breath flow in the tube.

According to some embodiments, the hydrophilic elements of the first and intermediate reservoirs may align an inner side of the inner walls of the first and intermediate reservoirs. According to some embodiments, the inner walls may include a plurality of port holes configured to allow liquids flowing in the channel of the reservoirs, to access, be absorbed and/or condensed out by the hydrophilic elements.

It is understood by one of ordinary skill in the art that the array of reservoirs may include reservoirs having their hydrophilic element differently positioned. For example, the first reservoir may include a hydrophilic element coextensive with the inner wall thereof, whereas the intermediate reservoir may include a hydrophilic element aligning the inner side of the inner wall thereof. For example, the first reservoir may include a hydrophilic element aligning the inner side of the inner wall thereof, whereas the intermediate reservoir may include a hydrophilic element coextensive with the inner wall thereof. For example the array of reservoirs may include a plurality of intermediate reservoirs some of which include a hydrophilic element aligning an inner side of the inner wall thereof and some of which including a hydrophilic element coextensive with the inner wall thereof.

According to some embodiments, the first and intermediate reservoirs include an outer wall configured to condense out liquids. According to some embodiments, the outer wall may include a hydrophilic material. The hydrophilic material may be a hydrophilic wicking material such as a porous plastic having a pore size ranging from approximately 5 microns to approximately 50 microns. Hence, liquids absorbed by the hydrophilic element may be condensed out through the outer wall of the reservoir.

According to some embodiments, the hydrophobic element of the first reservoir is disposed across the channel of the first reservoir at a proximal end thereof. Such configuration may ensure that any residual moisture still present in the breath sample will be prevented from reaching the medical device.

According to some embodiments, the array of reservoirs is attached to, embedded in or molded on the tube. According to some embodiments, the array of reservoirs is an integral part of the breath sampling tube. According to some embodiments, the array of reservoirs are add-ons, configured to be modularly connected to a breath sampling tube. For example, the array of reservoirs may be connected to the breath sampling tube, for instance through connectors. Alternatively, each of the first and intermediate reservoirs may be separately connected to the breath sampling tube, for instance through connectors.

According to some embodiments, the inner tube (the breath sampling tube interconnecting the reservoirs) is of a disposable nature.

According to some embodiments, the array of reservoirs may be of a disposable nature. According to some embodiments, the array of reservoirs may be reusable. According to some embodiments, each of the first and intermediate reservoirs may be separately reusable. This may enable to replace reservoirs at a different frequency. For example, reservoirs close to the patient end of the sampling tube may be replaced more often, than reservoirs further down the sampling line receiving only residual amounts of liquids. It is understood by one of ordinary skill in the art that such configuration may be of advantage, for example in home care settings, by maximizing the lifetime of the relatively expensive reservoirs while only replacing the sampling tube and/or replacing only parts of the reservoir array.

According to some embodiments, the array of reservoirs may include at least one intermediate reservoir. As used herein, the term “at least one” when referring to intermediate reservoirs may refer to 1, 2, 3, 4, 5 or more reservoirs. Each possibility is a separate embodiment.

According to some embodiments, the at least one intermediate reservoir may be disposed serially along the tube. For example the array of reservoirs may include at least two intermediate reservoirs disposed serially at sections along the tube. According to some embodiments, the array of reservoirs may include a plurality of intermediate reservoirs disposed serially along the tube. As used herein, the term “a plurality” when referring to intermediate reservoirs may refer to 5, 6, 7, 8, 9, 10, 15, 20 or more reservoirs. Each possibility is a separate embodiment.

According to some embodiments, the at least one intermediate reservoir may be disposed parallel along the tube. For example the array of reservoirs may include at least two intermediate reservoirs disposed at a parallel section along the tube. According to some embodiments, the array of reservoirs may include a plurality of intermediate reservoirs disposed parallel along the tube. As used herein, the term “a plurality” when referring to intermediate reservoirs may refer to 5, 6, 7, 8, 9, 10, 15, 20 or more reservoirs. Each possibility is a separate embodiment.

According to some embodiments, the at least one intermediate reservoir may include intermediate reservoirs being disposed parallel and serially along the tube. For example the array of reservoirs may include two intermediate reservoirs disposed at parallel sections along the tube and two additional parallel reservoirs disposed serially relative to the first pair of intermediate reservoirs. According to some embodiments, the array of reservoirs may include a plurality of intermediate reservoirs disposed at parallel and serial sections along the tube. As used herein, the term “a plurality” when referring to intermediate reservoirs may refer to 5, 6, 7, 8, 9, 10, 15, 20 or more reservoirs. Each possibility is a separate embodiment.

According to some embodiments, the number of intermediate reservoirs is modular. This may facilitate breath sampling lines having different lifetimes accommodating the intended use of the line. For example, breath sampling tubes for use with patients requiring long term monitoring of their respiratory gases, may include a relatively large number of intermediate reservoirs (e.g. 5-10 intermediate reservoirs) along the tube. Such increased number of reservoirs extend the life time of the tube and consequently reduce the need for frequent replacement of the breath sampling tube due to clogging and thus reduces both waste and costs. Alternatively, breath sampling tubes for use with subjects requiring only short term monitoring (e.g. subjects undergoing a breath test), may include a relatively small number of intermediate reservoirs (e.g. 1-5 intermediate reservoirs) along the tube, since the lifetime of the tube is up-front expected to be relatively short. The reduced number of reservoirs may sufficiently extend the lifetime of the tube to avoid a need for replacement while reducing the costs of production to a minimum.

Similarly, the number of reservoirs along the tube may differ based on the age of the patient. For example, breath sampling tubes for use with adults may include a relatively large number of intermediate reservoirs (e.g. 5-10 intermediate reservoirs) along the tube, whereas breath sampling tubes for use with children, infants and/or neonates may include a relatively small number of intermediate reservoirs (e.g. 1-5 intermediate reservoirs).

According to some embodiments, each reservoir may be distanced from its neighboring reservoir by a predetermined length. For example, the first reservoir may be distanced from a first intermediate reservoir by a distance of for example 1 cm, 2 cm, 5 cm, 10 cm, 20 cm, or 50 cm, any distance therebetween or more. Each possibility is a separate embodiment. For example, each intermediate reservoirs may be distanced from one another by a length of for example 1 cm, 2 cm, 5 cm, 10 cm, 20 cm, or 50 cm any distance therebetween or more. Each possibility is a separate embodiment. It is understood by one of ordinary skill in the art that the distance between the reservoirs along the tube may be different for different tubes. As a non-limiting example, the distance separating the reservoirs may be shorter for breath sampling tubes of one kind (e.g. tubes for use long term applications such as with intubated patients); and longer for other kinds of breath sampling tubes (e.g. tubes for short term applications). Similarly, the distance between the reservoirs along the tube may be fixed or variable. As a non-limiting example, the distance between the reservoirs at the proximal end of the tube, close to the medical device may be shorter than the distance between the reservoirs at a distal end of the tube or vice versa.

According to some embodiments, the array of reservoirs, sequentially absorbing/evaporating liquids from the breath sample as it flows through the breath sampling tube, enhances the overall evaporation of liquids from the tube, thus extending the lifetime of the tube. Therefore, under normal vapor pressure of water (P_{H2O}) conditions, the tube of the present disclosure can evaporate more liquids, thereby avoid blockage of the tube and in effect extend its lifetime.

According to some embodiments, the tube further comprises an inner conduit. At least a portion of the inner conduit may be non-cylindrical and configured to store liquids. According to some embodiments, the inner conduit is configured to permit gas flow along a central portion of the conduit and to store liquids along a surface of the conduit. According to some embodiments, the inner conduit may include a first lumen and a second lumen. The diameter of the first lumen may be larger than the diameter of the second lumen. According to some embodiments, the inner conduit may be adapted to collect liquids in the first lumen and to permit gas flow in the second lumen. According to some embodiments, the surface of the inner conduit comprises a hydrophilic material. According to some embodiments, the surrounding surface of the first lumen may be more hydrophilic than the surrounding surface of the second lumen.

There is provided, according to some embodiments, a breath sampling system including: a breath sampling tube having an array of reservoirs, and at least one connector. According to some embodiments, the array of reservoirs include a first reservoir proximate to a device end of the tube and at least one intermediate reservoir disposed along the tube. The first reservoir may include an inner wall defining a channel, a hydrophobic element and a hydrophilic element, as essentially described hereinabove. The at least one intermediate reservoir may include an inner wall defining a channel and a hydrophilic element, as essentially described hereinabove. According to some embodiments, the array of reservoirs is configured to allow unabsorbed water vapor to proceed through the at least one intermediate reservoir while essentially blocking unabsorbed water vapor from passing through the first reservoir.

There is provided, according to some embodiments, a method including forming a breath sampling tube having an array of reservoir. According to some embodiments, the array of reservoirs include a first reservoir proximate to a device end of the tube and at least one intermediate reservoir disposed along the tube. The first reservoir may include an inner wall defining a channel, a hydrophobic element and a hydrophilic element, as essentially described hereinabove. The at least one intermediate reservoir may include an inner wall defining a channel and a hydrophilic element, as essentially described hereinabove. According to some embodiments, the array of reservoirs is configured to allow unabsorbed water vapor to proceed through the at least one intermediate reservoir while essentially blocking unabsorbed water vapor from passing through the first reservoir.

According to some embodiments, at least part of the tube is formed of a material configured to evaporate water.

According to some embodiments, the method comprises attaching, embedding or molding the array of reservoirs on the tube. According to some embodiments, the array of reservoirs may be formed as an integral part of the breath sampling tube. Alternatively, the array of reservoirs may be connected to the breath sampling tube, for example through connectors. Alternatively, each of the first and intermediate reservoirs may be separately connected to the breath sampling tube, for example through connectors.

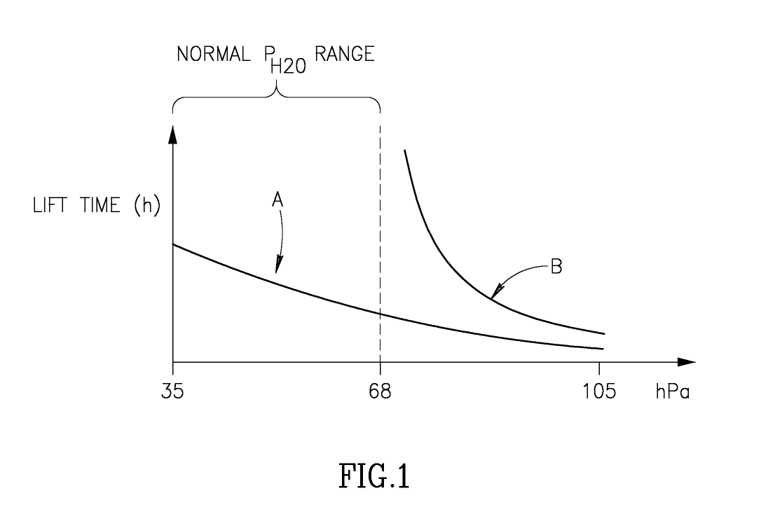

Reference is now made to FIG. 1, which schematically illustrates the lifetime of tube lines against the vapor pressure of water (P_{H2O}) in prior art tube lines (A), and in the tube lines disclosed herein (B), according to some embodiments. It is understood that as the vapor pressure (humidity) increases the life time of the sampling tube decreases. The normal P_{H2O} typically ranges from about 35 hPa to about 70 hPa, however P_{H2O} values as high as 105 hPa can also occur.

As seen in FIG. 1, the tube disclosed herein, comprising an array of reservoirs, has an extended life time due to the enhanced absorption and/or evaporation achieved through the array of intermediate reservoirs and through the first reservoir.

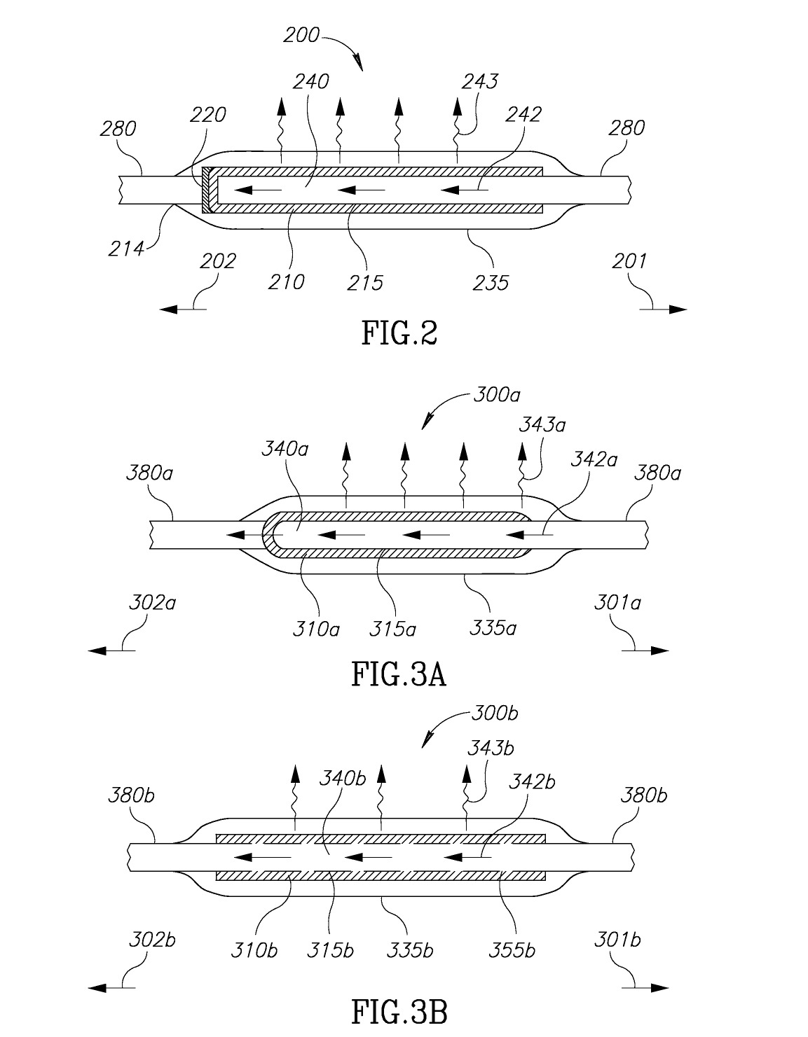

Reference is now made to FIG. 2, which schematically illustrates a first reservoir 200, according to some embodiments. First reservoir 200 includes a proximal end 202 (device end) configured to be connected proximate to a medical device, such as a capnograph (not shown), and a distal end 201. First reservoir 200 has an inner wall 215 defining a channel 240 arranged to facilitate fluid flow communication with breath sampling tube 280 thus allowing breath samples to flow undisturbed in the direction of the medical device, as illustrated by arrows 242. Coextensive with inner wall 215 is hydrophilic element 210 configured to absorb and/or condense out water vapor from the breath samples flowing in channel 240. The outer wall 235 of first reservoir 200 is made from a hydrophilic material configured to condense out liquids as illustrated by arrows 243. First reservoir 200 further includes a hydrophobic element 220 disposed across channel 240 at proximal end 202, close to the medical device. Hydrophobic element 220 is configured to repel water away from a breath sample outlet 214 thereby preventing any residual moisture still present in the breath sample from reaching the medical device. Optionally a connector (not shown) may be molded on or otherwise attached to proximal end 202 of first reservoir 200 enabling a direct connection of first reservoir 200 to the medical device.

Reference is now made to FIG. 3A, which schematically illustrates an intermediate reservoir 300a, according to some embodiments. Intermediate reservoir 300a includes a proximal end 302a and a distal end 301a. Intermediate reservoir 300a further includes an inner wall 315a defining a channel 340a arranged to facilitate fluid flow communication with breath sampling tube 380a thus allowing breath samples to flow undisturbed in the direction of the medical device, as illustrated by arrows 342a. Coextensive with inner wall 315a is hydrophilic element 310a configured to absorb and/or condense out water vapor from the breath samples flowing in channel 340a. The outer wall 335a of intermediate reservoir 300a is made from a hydrophilic material configured to condense out liquids as illustrated by arrows 343a. Intermediate reservoir 300a is devoid of a hydrophobic element. Thus any residual moisture still present in the breath sample can undisturbedly exit proximal end 302a of intermediate reservoir 300a and continue its flow in breath sampling tube 380a extending from proximal end 302a of intermediate reservoir 300a. Intermediate reservoir 300a may optionally include connectors (not shown) configured to modularly connect intermediate reservoir 300a to breath sampling tube 380a.

Reference is now made to FIG. 3B, which schematically illustrates an intermediate reservoir 300b, according to some embodiments. Intermediate reservoir 300b includes a proximal end 302b and a distal end 301b. Intermediate reservoir 300b further includes an inner wall 315b defining a channel 340b arranged to facilitate fluid flow communication with breath sampling tube 380b thus allowing breath samples to flow undisturbed in the direction of the medical device, as illustrated by arrows 342b. Aligning an inner side of inner wall 315b is hydrophilic element 310b configured to absorb and/or condense out water vapor from breath samples flowing in channel 340b. Inner wall 315b includes a plurality of port holes 355b configured to allow liquids flowing in channel 340b to access hydrophilic elements 310b. The outer wall 335b of intermediate reservoir 300b is made from a hydrophilic material configured to condense out liquids as illustrated by arrows 343b. Intermediate reservoir 300b is devoid of a hydrophobic element. Thus any residual moisture still present in the breath sample can undisturbedly exit proximal end 302b of intermediate reservoir 300b and continue its flow in breath sampling tube 380b extending from proximal end 302b of intermediate reservoir 300b in direction of the medical device. Intermediate reservoir 300b may optionally include connectors (not shown) configured to modularly connect intermediate reservoir 300b to breath sampling tube 380b.

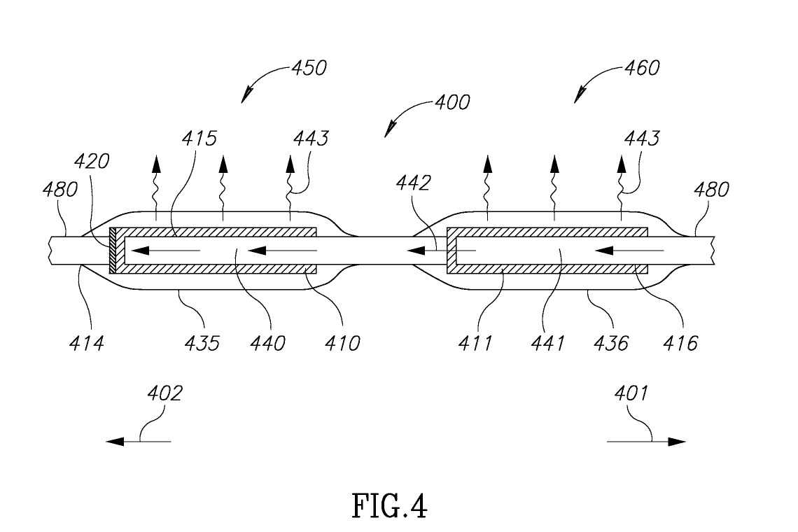

Reference is now made to FIG. 4, which schematically illustrates a breath sampling line 400 including an array of reservoirs, according to some embodiments. Sampling line 400 includes a proximal end 402 (device end) configured to be connected proximate to a medical device, such as a capnograph (not shown), and a distal end 401. At proximal end 402 of sampling line 400 is a first reservoir 450 and distally thereto at least one intermediate reservoir, here illustrated as intermediate reservoir 460, however sampling line 400 may include additional intermediate reservoirs, as discussed hereinabove. First reservoir 450 has an inner wall 415 defining a channel 440 arranged to facilitate fluid flow communication with breath sampling tube 480 and with channel 441 defined by inner wall 416 of intermediate reservoir 460, thus allowing breath samples to flow undisturbed in the direction of the medical device, as illustrated by arrows 442. Coextensive with inner walls 415 and 416 are hydrophilic elements 410 and 411 respectively, configured to absorb and/or condense out water vapor from the breath samples flowing in channels 440 and 441. Hydrophilic elements 410 and 411 are here illustrated as being coextensive with inner walls 415 and 416, however other configurations, such as, but not limited to, that described in FIG. 3B are also applicable and as such fall within the scope of the disclosure. First and intermediate elements 450 and 460 include outer walls 435 and 436 made from a hydrophilic material configured to condense out liquids as illustrated by arrows 443.

First reservoir 450 only further includes a hydrophobic element 420 disposed across channel 440 at proximal end 402, close to the medical device. Hydrophobic element 420 is configured to repel water away from a breath sample outlet 414 thereby preventing any residual moisture still present in the breath sample from reaching the medical device.

Optionally a connector (not shown) may be molded on or otherwise attached to proximal end 402 of first reservoir 450 enabling a direct connection of first reservoir 450 to the medical device. First and intermediate reservoirs 450 and 460 may also include connectors on both sides thereof enabling modulatory connection of the reservoirs to sampling tube 480. Alternatively, connectors may be molded on or otherwise attached to both sides of the entire array of reservoirs or parts thereof, likewise enabling modulatory attachment of the reservoirs to breath sampling tube 480.

Referring to FIG. 4, the following is a description of the operation of sampling line 400 according to some embodiments. A patient is connected to a respiratory apparatus or to some other ventilation means through a cannula or a patient airway tube, collectively referred to as a respiratory output device (not shown). Samples of exhaled breath from the patient, which may include liquid secretions such as blood, mucus, water, medications, and the like, are sucked into sampling tube 480 from the respiratory output device, typically by means of negative pressure supplied by a pumping element (not shown) connected to breath sampling tube 480. The breath sample, including the liquid secretions, initially pass through the at least one intermediate reservoir, here illustrated as intermediate reservoir 460 where moisture is extracted from the exhaled breath by hydrophilic element 411. A first of the at least one intermediate elements may advantageously be positioned as close as possible to the respiratory output device so as to immediately try to counteract the effects of the liquids in the exhaled breath samples which may contribute to clogging in sampling tube 480. Although intermediate reservoir 460 may able to extract a good portion of the moisture and liquids, significant amounts may remain in the exhaled breath samples which may hamper the accurate monitoring and analysis of the samples by the measurement sensor in addition to possibly blocking the path of the flow of the samples in sampling line 400. However, first reservoir 450 positioned proximate to the medical device on the one hand enhance the evaporation of the liquids through hydrophilic element 410 and on the other hand repels any residual moisture away from breath sampling outlet 414 by means of hydrophobic element 420. Hence, whereas intermediate reservoir 460 is configured to allow unabsorbed water vapor to proceed therethrough, first reservoir 450 blocks unabsorbed water vapor from passing therethrough. Such configuration prevents water from reaching the medical device on the one hand, while on the other hand avoids clogging of breath sampling tube 480 due to accumulation of liquids along breath sampling tube 480. It may be advantageous to add more than one intermediate reservoir, each reducing the amount of water vapor reaching first reservoir 450. The number of intermediate reservoirs needed depends on the length and desired lifetime of the tube as well as the age of the patient to whom it is connected.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. A breath sampling tube comprising an array of reservoirs, said array of reservoirs comprising a first reservoir proximate to a device end of said tube and at least one intermediate reservoir; said first reservoir comprising an inner wall defining a channel, a hydrophobic element and a hydrophilic element; and said at least one intermediate reservoir comprising an inner wall defining a channel and a hydrophilic element; wherein said array of reservoirs is configured to allow unabsorbed water vapor to proceed through said at least one intermediate reservoir while essentially blocking unabsorbed water vapor from passing through said first reservoir.

2. The tube according to claim 1, wherein said at least one intermediate reservoir is devoid of a hydrophobic element.

3. The tube according to claim 1, wherein said hydrophilic elements of said first and intermediate reservoirs are coextensive with said inner wall of said first and intermediate reservoirs.

4. The tube according to claim 3, wherein said hydrophilic elements comprises a hydrophilic material transiting the inner wall.

5. The tube according to claim 4, wherein said hydrophilic material is of a thickness that maximizes water vapor while minimizing disruption of breath flow in said tube.

6. The tube according to claim 1, wherein said hydrophilic elements of said first and intermediate reservoirs aligns an inner side of said inner walls of said first and intermediate reservoirs.

7. The tube according to claim 6, wherein said inner walls comprise a plurality of port holes configured to allow breath, flowing in said channel of said first reservoir and of said intermediate reservoir, to access said hydrophilic elements.

8. The tube according to claim 1, wherein said hydrophobic element of said first reservoir is disposed across said channel of said first reservoir at a proximal end thereof.

9. The tube according to claim 1, wherein the array of reservoirs is attached to, embedded in or molded on said tube.

10. The tube according to claim 1, comprising at least two intermediate reservoirs disposed serially along said tube.

11. The tube according to claim 1, comprising at least two intermediate reservoirs disposed parallel along said tube.

12. The tube according to claim 1, comprising a plurality of intermediate reservoirs.

13. The tube according to claim 1, wherein said at least one intermediate reservoir is configured to reduce the amount of water vapor reaching said first reservoir.

14. The tube according to claim 1, wherein said array of reservoirs enhances the evaporation of liquids from said tube, thereby extending the life time of said tube.

15. The tube according to claim 1, further comprising an inner conduit.

16. The tube of claim 15, wherein said inner conduit is configured to permit gas flow along a central portion of said conduit and to store liquids along a surface of said conduit.

17. The tube of claim 15, wherein the surface of said inner conduit comprises a hydrophilic material.

18. A breath sampling system comprising:
a breath sampling tube comprising an array of reservoirs, said array of reservoirs comprising a first reservoir proximate to a device end of said tube and at least one intermediate reservoir; said first reservoir comprising an inner wall defining a channel, a hydrophobic element and a hydrophilic element; and said at least one intermediate reservoir comprising an inner wall defining a channel and a hydrophilic element; wherein said array of reservoirs is configured to allow unabsorbed water vapor to proceed through said at least one intermediate reservoir while essentially blocking unabsorbed water vapor from passing through said first reservoir; and
at least one connector.

19. A method comprising forming a breath sampling tube comprising an array of reservoirs, the array of reservoirs comprising a first reservoir proximate to a device end of the tube and at least one intermediate reservoir; the first reservoir comprising an inner wall defining a channel, a hydrophobic element and a hydrophilic element; and the at least one intermediate reservoir comprising an inner wall defining a channel and a hydrophilic element; wherein the array of reservoirs is configured to allow unabsorbed water vapor to proceed through the at least one intermediate reservoir while essentially blocking unabsorbed water vapor from passing through the first reservoir.

20. The method of claim 19, wherein at least part of the tube is formed of a material configured to evaporate water.
